(19) United States
(12) Patent Application Publication
(10) Pub. No.: US 2005/0064040 A1
(43) Pub. Date: Mar. 24, 2005

(54) Processes for improving binding capability and biological digestability

(76) Inventor: Anders Aksnes, Bergen (NO)

Correspondence Address: Francis C. Hand;Carella Byrne Bain Gilfillan Cecchi Stewart & Olst, 5 Becker Farm Road, Roseland, NJ, 07068, US

(21) Appl. No.: 10/500,805

(22) PCT Filed: Jan. 7, 2003
(86) PCT No.: PCT/NO03/00002

(30) Foreign Application Priority Data
Jan. 7, 2002 (NO) 20020047

(51) Int. Cl. A61K 35/60

(52) U.S. Cl. 424/548; 424/520; 426/635

(57) ABSTRACT

The present invention describes processes for obtaining a fish product with improved binding capabilities. Gelatine is liberated from collagen in connective tissues, skin and bones that is naturally present in the fish raw material as the product is produced from. Further, the present application describes processes for improving the digestibility of bone bounded minerals in feeds. The result is improved biological digestibility of calcium and phosphorus.

### FIELD OF THE INVENTION

The present invention relates to a process for improving the binding capability of a fish material. The invention also relates to a process for improving the biological digestibility of a feed product made of a raw material. In a further aspect the invention also relates to process for increasing both the binding capability and the biological digestibility of a product, and to a product manufactured in accordance with this process.

More specifically, the present invention relates to a line of action to processing of fish or part of fish, for liberation of gelatine, and/or for improvement of the biological digestibility of these and thus for reduction of effluents and improvement of the environment.

### BACKGROUND FOR THE INVENTION

In the production of feed for fish and animals it is important that the various ingredients are hold together in one larger feed particle. Binding agents are used for this purpose, and these are often carbohydrate containing compounds. These binding agents may be indigestible for the animal or fish that are fed.

Industrial feeding is dependent on good physical characteristics of the feed particles. One needs feed particles that are kept together and not broken at transport, storage, handling and feeding. In some type of feeds (for instance feed for eels) it is important that the feed are like a dough and that the eel can eat small bits of the feed particles in a way that keep the rest of the feed particle as an unbroken particle. This will depend on the characteristics of the feed ingredients and the binder that are used. Good binding capability is also important in production of fish products as for instance fish balls and cakes.

An object of the present invention is to increase the binding properties of a product without the addition of a binding agent. The binding properties are improved by activating inherent binding capabilities already inherent in the raw material. Such a material with increased binding properties may open new possibilities for the use of new fish species for manufacturing of food product for human consumption and also for the production of improved feed products.

The processes and products according to the present invention reduce or eliminate the requirements for the supplementing of binding agents.

Fish meal is an important ingredient in feed for fish and animals, and fish feed may contain 30-50% of the total dry matter as fish meal. Fish meal is produced from whole fish and parts of fish that contain collagen rich protein; bone, skin, tendons and connective tissues.

Without being bound to a specific theoretical mechanism it is believed that the inherent binding capability of the raw material is activated or induced by a conversion of collagen to a gelatinous component. It is thus preferred that the raw product contains collagen.

Gelatine is a mixture of proteins and may be liberated from collagen from these tissues by chemical or thermal methods.

The prior art discloses methods for isolation and extracting of gelatine from a sample. Most often, gelatine is liberated by heating in hot water. The gelatine that can be liberated by known methods has a characteristic composition of amino acids and special properties by making a jelly at low concentrations in water.

A further object of the present invention relates to a process for making minerals that are present in bones as crystals more soluble and thus digestible for the consumer.

Raw fish bones contains about 50% dry matter, and thereof about 25% collagen, 5-10% lipids and about 20% ash. More than half of the ash is calcium and phosphorus and are used to build the bone structure. These minerals are present in the bones as crystals of hydroxyapatite (Ca₅(PO₄)₃(OH)) and calcium carbonate (CaCO₃). These are stable chemical compounds that are difficult to liberate in the intestinal tract of animals and fish. The digestibility of minerals from bone is therefore low (20-50% digestibility of phosphorus in salmon) and a significant part of these minerals are effluents to the environment as indigestible nutrients. Increased level of phosphorus (as phosphate) in the environment causes good conditions for growth of microorganisms. The governmental rules are therefore strict for the maximum allowable content of phosphorus in the feed. If at the same time, the digestibility of the phosphorus is low (that are the situation in feed for fish), the biological availability may be too low. This may result in deceases due to shortage, deformation in the skeleton or fish death.

It is known that water soluble minerals have good digestibility in fish feed for instance (>90% for phosphorus in salmon), but governmental regulations for total level of certain minerals limit the possibility to add minerals with good digestibility. Especially this is the case for phosphorus. It is therefore a demand and need for minerals that are naturally present in the raw materials, are available in a chemical form that are easily digested.

### DESCRIPTION OF THE INVENTION

Connective tissues, skin and bone from fish raw materials, contain a potential for liberation of gelatine. This will have binding properties that will improve the physical characteristics in processing of feed and fish products by keeping the particles in the product together. Further, bone contains indigestible minerals. These may be dissolved at processing and thus make them digestible for the organism to be fed. In processing of fish bones, liberated gelatine may be combined with dissolution of bone-bounded minerals and thus make the minerals soluble in water with improved digestibility. In this way, the same fish bones are used for both improvement of binding capacity by increased gelatine content and increased digestibility of bone-bounded minerals, mainly calcium and phosphorus.

The invention may also be used for other applications where binding property of the product to be processed needs binding capacity. This may be the case when fish raw materials are used directly in feed processing or in products for human consumption where the fish raw materials are mixed with other ingredients as for the production of for instance fish balls or fish cakes.

Solubilization of gelatine from collagen protein in skin and bone is in the present invention utilised in the same production and are therefore present in the same products that are produced.

As preferred raw material for application of the invention may be fish, by-products from the fish industry or intermediate products in fish processings. By-products from fish processing normally contain high levels of skin and bones after filleting or deskinning. As an example for intermediate products that may be used, is bone particles from fish meal processing that are collected by sieving before milling of the fish meal. In this case, gelatine has to be liberated by chemical or thermal processing. Liberated gelatine with binding properties is then returned to the fish meal and dried and milled as usually in the production. This produces a fish meal with improved binding property, and the feed pellets are kept together by the gelatine that is present. Liberation of gelatine from fish bone may be performed by heating in water, high pressure cooking or by repeated or continuous extraction by hot processing liquids (for instance stick water).

By-products from the fish industry are also used as raw material for fish meal processing. For this kind of raw material, gelatine with binding property may be liberated by chemical or thermal methods before it is used as raw material in fish meal processing. In this way a fish meal with gelatine is obtained and thus a fish meal with improved physical properties for feed production.

The liberation of gelatine from fish bone results in a matrix of minerals where protein and other carbon-containing compounds are removed. These minerals are however still present as hydroxyapatite and calcium carbonate and have low digestibility in some biological organisms (e.g. fish). According to the present invention these minerals are made soluble and thus dissolved in the aqueous phase. This can be conducted by chemical treatment: acid, alkaline or high ion-concentration (i.e. salts). The minerals will be present as dissolved ions and thus have a high digestibility. When the dissolved minerals are returned to the fish raw material or intermediate products in the fish meal processing, a higher digestibility of these minerals will be obtained.

By sieving of unmilled fish meal, bone particles are removed and thus most of the minerals that are present in stable chemical compounds and thus have low digestibility. By dissolving the mineral complexes and return them, a significant increase in digestibility of these minerals are obtained. The ultimate goal is a feed ingredient from fish raw material where the fraction of indigestible minerals are reduced and the fraction of digestible minerals are significantly increased.

### Experimental Section

### EXAMPLE 1

Preparation of Gelatine-Sample from Fishbone

Bone from salmon back was rinsed for muscle tissue, added 3 parts of water and heated for 10 hours at 100° C. The water phase was separated from not solubilized bone residue by a 1 mm sieve and then filtered trough paper to remove small amounts of sludge. As shown in table 1, the phase mainly consists of solubilized protein (gelatine) that is liberated from the salmon bones. The unsolubilized residues looked like perforated spinal cords and that were crispy and easy to break between the fingers. As also shown in table 1, this residue was mainly ash.

TABLE 1
Chemical composition of fish bone and fractions after heating.
All values are in % of the samples.

| | Dry matter | Protein | Lipid | Ash | Calcium | Phosphorus |
|---|---|---|---|---|---|---|
| Fish bone | 52 | 20 | 5 | 26 | 10 | 5 |
| Water phase | 11 | 10 | | <1 | | |
| Residues | 36 | 3 | | 30 | | |

Test for Binding Property in Fish Meal

A fish meal from Norway was added to the above prepared water phase in an amount equivalent to 2.5% of dry matter. This is about equal to the amount of dry matter that was liberated from the bones adjusted to the whole fish weight. Water was added to a total water content of 25%. The mixture was blended in a mortar. A control group was treated similarly except that water instead of gelatine extract was added to the fish meal.

The fish meal had the following chemical composition before addition of water or solubilised gelatine from fish bone:

| | |
|---|---|
| Water | 9.8% |
| Protein | 69.5% |
| Lipid | 11.4% |
| Ash | 9.8% |

Pellets were made by pressing 1 gram samples of the mixtures in a steel cylinder with a piston. Two series of pellets were produced using two different piston pressures, 57 kiloponds and 115 kiloponds. The compressed pellets were about 12.5 mm in diameter and 7 mm height. The pellets were dried at 40° C. over night.

The quality of the pellets was tested in a Pharma Test model PTB311P. This works by putting a continuously increased pressure on the pellets and the pressure where the pellets are broken into pieces is registered. The equipment has a lower limit of 1 kilopond and a n upper limit of 30 kiloponds. Table 2 a shows the power needed to break the pellets from the control group and the group added solubilized gelatine in pellets produced by using a pressure in the cylinder of 115 kiloponds. Each group was tested with the cylindrical pellets in upright and lying position. Similar values are given in Table 2b for pellets produced by 57 kiloponds.

TABLE 2
Number of fish meal pellets tested, and the power needed to break the pellets
2a. Fish meal pellets produced at 115 kiloponds

| | Pellets lying | | | Pellets upright | | |
|---|---|---|---|---|---|---|
| | Number | Kiloponds | Standard | Number | Kiloponds | Standard |
| Added | tested | for breach | deviation | tested | for breach | deviation |
| Water (control) | 6 | 16.0 | 1.3 | 6 | <1 | |
| Solubilised gelatin | 5 | >30 | | 9 | 1.8 | 0.2 |

2b. Fish meal pellets produced at 57 kiloponds

| | Pellets lying | | |
|---|---|---|---|
| | Number | Kiloponds | Standard |
| Added | tested | for breach | deviation |
| Water (control) | 10 | 13.4 | 0.9 |
| Solubilised gelatine | 5 | >30 | |

As clearly shown in Table 2, the pellets that contain solubilized gelatine needs higher pressure before the pellets are broken and thus that solubilized gelatine increase the binding capacity. An other significant difference between the groups is that the pellets from control group was broken into many small pieces, while pellets produced from fish meal added solubilized gelatine were broken into in a much fewer greater pieces.

### EXAMPLE II

Test for Binding Capacity in a Feed Mixture.

A similar experiment as described in example I was carried out with a feed mixture instead of fish meal. All additions and treatments were otherwise as described in example I. The feed mixture had the following content before addition of water or solubilised gelatine from fish bones:

| | |
|---|---|
| Fish meal | 65% |
| Cellulose | 15% |
| Oil | 10% |
| Corn starch | 7% |
| Vitamins and Minerals | 2.5% |
| Lecithin | 0.5% |

The chemical composition of the feed mixture was: dry matter: 92.3%; protein, 46.6%, carbohydrate, 20.4% and lipid, 15.7%.

The results from the test to measure the power needed to break the pellets produced from the feed mixture are given in Table 3.

TABLE 3
Number of pellets tested, and the power needed to break the pellets.
3a. Pellets from feed mixture produced at 115 kiloponds.

| | Pellet lying | | | Pellet upright | | |
|---|---|---|---|---|---|---|
| | Number | Kiloponds | Standard | Number | Kiloponds | Standard |
| Added | tested | for breach | deviation | tested | for breach | deviation |
| Water (control) | 5 | >30 | | 5 | <1 | |
| Solubilised gelatine | 5 | >30 | | 5 | 1.5 | 0.5 |

3b. Pellets from feed mixture produced at 57 kiloponds

| | Pellet lying | | |
|---|---|---|---|
| | Number | Kiloponds | Standard |
| Added | tested | for breach | deviation |
| Water (control) | 5 | 18.3 | 2.5 |
| Solubilised gelatine | 5 | >30 | |

### EXAMPLE III

Solubilization of Minerals from Fish Bone

Minerals in bone material may be separated from collagenous bone proteins by acid or alkaline treatment. The dissolved minerals are water soluble minerals ions and thus more digestible for animals and fish. Example 3 is carried out to show that acid treatment of fish bones will separate collagenous protein from minerals present in the bones and that the liberated minerals are soluble in water. The collagenous proteins in the residues may thereafter be treated to liberate and dissolve gelatine to obtain improved binding capacity when used in a feed mixture.

Raw cleaned fish bones from salmon were added 3 parts water and ½ part 36% hydrochloric acid. The mixture was stirred cautiously for 12 hours at 10° C. The water soluble phase was separated by sieving. Table 4 shows the chemical composition of the bone raw material, the water phase and bone residues. The bone residues were washed 4 times with equal amounts of water.

TABLE 4
Chemical composition of bone raw material and fractions after
acid treatment.
The values are given as dry matter in kilograms in the experiment.

| | Dry | | | | |
|---|---|---|---|---|---|
| | matter | Protein | Ash | Calcium | Phosphorus |
| Bone raw | 4.70 | 1.52 | 2.18 | 0.78 | 0.40 |
| material | | | | | |
| Water phase | 2.61 | 0.05 | 1.80 | 0.55 | 0.28 |
| Residue | 1.51 | 1.28 | 0.07 | 0.02 | 0.01 |

The experiment shows clearly that bone proteins that are separated from the minerals as proteins are present in the undissolved residue while the minerals are present in the water phase. By the process according to the present invention the minerals originally present in the bones are made more soluble and thus more digestible. At the same time bone collagen is isolated and may be treated for liberation of soluble gelatine that may be utilised to improve binding capacity in feeds and fish products.

1. A process for improving the binding capability of a fish material, characterized in that a collagen containing fish material is subjected to a gelatine liberation step in order to improve the inherent binding capability of said fish material.

2. A process in accordance with claim 1, characterized in that the collagen containing fish material is selected from the group comprising connective tissues, skin and bones from fish.

3. A process in accordance with claim 1, characterized in that said fish material is selected from the group comprising a) bone from fish by-products, b) skin from fish by-products, c) whole fish for processing to feed, d) whole fish for processing to feed ingredient and e) bone containing intermediate products from fish meal processing.

4. A process in accordance with claim 1, characterized in that said gelatine liberation step is a physical and/or chemical conversion of insoluble collagen in said collagen containing fish material to a water soluble gelatinous material.

5. A process in accordance with claim 1, characterized in that the gelatinous material is not isolated or extracted from the fish material.

6. A process in accordance with claim 1, characterized in that the gelatine liberation step is conducted by adding an aqueous liquid to the fish material, and thereafter heating the mixture of fish material and liquid to at least 50° C. for at least 3 hours.

7. A process in accordance with claim 6, characterized in that the gelatine liberation step is conducted by heating the mixture at about 100° C.

8. A process in accordance with claim 6, characterized in that the gelatine liberation step is conducted by heating the mixture in about 10 hours.

9. A process in accordance with claim 6, characterized in that the ratio of fish material to water is in the range of 1:10 to 1:1, preferable 1:5 to 1:2, most preferable about 1:3, based on weight/weight.

10. A process in accordance with claim 4, characterized in the liberation of gelatine is conducted by high pressure cooking.

11. A process in accordance with claim 4, characterized in that the liberation of gelatine is conducted by repeated or continuous extraction by hot processing.

12. A process in accordance with claim 4, characterized in that the liberation of gelatine is conducted by repeated or continuous extraction by hot stickwater from the fish meal processing.

13. A process in accordance with one of the preceding claims, characterized in that said gelatine liberation step is included in the conventional processing of a fish material to a fish meal product.

14. A process in accordance with one of the preceding claims, characterized in that said gelatine liberation step is included in the conventional processing of a fish material to a fish feed product.

15. A process in accordance with one of the preceding claims, characterized in that said gelatine liberation step is included in the conventional processing of a fish material to a fish product for human consumption such as for instance fish forcemeat, fish balls or fish cakes.

16. A process for improving the biological digestibility of a feed product made of a raw material comprising as a minor or substantial fraction of bone tissues, characterized in that the raw material is subjected to a process of for gelatine liberation wherein the proteins in the bone tissue are made digestible.

17. A process for improving the biological digestibility of a feed product made of a raw material comprising as a minor or substantial fraction of bone tissues, characterized in that the raw material is subjected to a process of acid or alkaline treatment wherein minerals in the bone tissue are made soluble.

18. A process in accordance with claim 17, characterized in that the treatment is conducted by the addition of hydrochloric acid.

19. A process in accordance with claim 18, characterized in that the concentration of said hydrochloric acid is 0.1 to 10 M, preferable 0.5 to 3M.

20. A process in accordance with one of the claims 17-19, characterized in that the solubilization of minerals from bones are conducted as a process step in the process line of a conventional manufacturing of a feed product.

21. A process in accordance with one of the claims 17-19, characterized in that a fraction containing soloubilized minerals is added as an ingredient to a feed product.

22. A process in accordance with one of the claims 17-21, characterized in that said bone tissue is fish bones.

23. A process for increasing the binding capability and the biological digestibility of a product, characterized in that a collagen and bone tissue containing raw material is subjected to both a gelatine liberation step in order to improve the inherent binding capability and a process of acid or alkaline treatment wherein minerals in the bone tissue are made soluble.

24. A process in accordance with claim 23, characterized in that a process according to one of the claims 1 to 16 is used in combination with a process according to claim 17 to 21.

25. A process in accordance with one of the claims 22 and 23, characterized in that the collagen and bone tissue containing raw material is a fish material.

26. A process in accordance with one of the claims 22-25, characterized in that raw material get a higher temperature load than equivalent to 90° C. in more than 40 minutes, and that a hot processing liquid is used to extract gelatine from the collagen source.

27. A product with improved binding capability and biological digestibility, characterized in that the product is manufactured of a collagen and bone tissue containing raw material which is subjected to both a gelatine liberation step in order to improve the inherent binding capability and a process of acid or alkaline treatment wherein minerals in the bone tissue are made soluble.

28. A product in accordance with claims 27, characterized in that the collagen and bone tissue containing raw material is a fish material.

29. A product in accordance with one of the claims 27 or 28, characterized in that said product is manufactured by a process according to one of the claims 1 to 16 in combination with a process according to claim 17 to 21.

30. A product in accordance with one of the claims 27-29, characterized in that the product is used as a supplement ingredient in the manufacturing of a pharmaceutical, veterinary, food and cosmetic products.
